(19) United States
(12) Patent Application Publication
(10) Pub. No.: US 2025/0281308 A1
(43) Pub. Date: Sep. 11, 2025

(54) PROSTHESIS COVER AND METHOD FOR PRODUCING A PROSTHESIS COVER

(71) Applicant: OTTO BOCK HEALTHCARE PRODUCTS GMBH, Wien (AT)

(72) Inventors: Walter LUNZER, Wien (AT); Sonja WAGNER, Wien (AT); Angelika WILLCOCK, Wien (AT); Andreas HOGH, Wien (AT); Johannes BISCHOF, Wien (AT)

(21) Appl. No.: 18/860,109

(22) PCT Filed: Apr. 26, 2023
(86) PCT No.: PCT/EP2023/060999

§ 371 Date: Oct. 25, 2024

(30) Foreign Application Priority Data
May 6, 2022 (DE) 10 2022 111 268.3

(51) Int. Cl. A61F 2/60; A61F 2/50

(52) U.S. Cl. CPC A61F 2/60; A61F 2002/5001; A61F 2002/5007; A61F 2002/607

(57) ABSTRACT

The invention relates to a prosthesis cover having at least one fastening device (20) for securing the prosthesis cover (1) to a prosthesis component (2), and a flexible main body (10) which has an inner side (11) facing the prosthesis component (2) in the applied state and an outer side (12) opposite the inner side (11), on which outer side the at least one fastening device (20) is arranged, wherein at least one structural element (30) protruding from the outer side (12) is applied to the outer side (12) in an additive manufacturing process.

The invention relates to a prosthesis cover comprising at least one fastening device for fixing the prosthesis cover to a prosthesis component and a flexible main body having an inner side, which faces the prosthesis component in the placed-on state, and an outer side, which is situated opposite the inner side and on which the at least one fastening device is arranged. The invention also relates to a process for producing a prosthesis cover comprising at least one fastening device for fixing the prosthesis cover to a prosthesis component and a flexible main body, having an inner side, which faces the prosthesis component in the placed-on state, and an outer side, which is situated opposite the inner side and on which the at least one fastening device is arranged.

Prosthesis covers or prosthesis cosmetics serve to clad a prosthesis component with the aim, among other things, of achieving as natural as possible an appearance of the replaced limb and in order to protect the prosthesis component against external influences. Furthermore, prosthesis cosmetics and prosthesis covers protect the surrounding area against direct contact with the prosthesis component and furthermore have an esthetic component.

Prosthesis covers can be produced from a foam material, the manufacture of such prosthesis covers being complex. The production involves adapting a main body, made of a foam material and having a cavity for receiving the prosthesis component, to the respective patient by removing material. The material is removed predominantly by sanding or by other cut-off methods and requires an orthopedic technician to have a lot of experience and spend a relatively long time. Owing to the rough surface produced during the sanding operation, such prosthesis cosmetics cannot yet be used acceptably, and rather it is necessary to draw a further covering, for example made of an elastic fabric material, over the outer surface.

In addition to making prosthesis covers from a foam material, there exist bowl-like prosthesis covers, which cover the functional elements of a prosthesis and have a smooth outer side. These prosthesis covers consist of plastic, a composite material or a metal and have a rather technical appearance.

DE 10 2018 127 117 A 1 discloses a prosthesis cladding and a process for producing it, in the course of which a dimensionally stable main body having a cavity for receiving a prosthesis component is created in an additive production process. The main body is formed in one piece and has an open cross section with abutting edges oriented in the longitudinal extent of the prosthesis cladding. The prosthesis cladding can be curved along its longitudinal extent. The main body may have a mesh structure and correspond substantially to the contour of the limb replaced by the prosthesis component.

US 2007/0 162 154 A 1 discloses a prosthesis cover comprising a hollow, flexible main body, which has a substantially cylindrical basic shape. A seam extends from a lower opening to the upper opening, in order to be able to place the cover around the prosthesis component. The closure device provided is for example a hook-and-loop closure. The material of the main body is neoprene.

DE 10 2018 132 640 A 1 relates to an elastic sheet-like material and to a process for producing it, in the course of which a functional element is connected to the sheet-like material and at least one limiting element is arranged between the functional element and the sheet-like material or on the opposite side of the sheet-like material from the functional element. The limiting element reduces the elasticity of the sheet-like material and increases the adhesion of the functional element owing to the reduced relative movement when the sheet-like material is used. The functional element can be applied to the sheet-like material or to the limiting element in an additive production process.

WO 2022/024114 A1 relates to a system and a process for 3D printing of textiles, in the course of the process an array of nozzles for applying the material to be printed being arranged above a printed fabric. Computer control is used to activate the nozzles, in order to produce the desired shape of an object on the textile. To harden the material, it is irradiated with UV light or heated.

The individual manufacture of prosthesis covers is also associated with high complexity, for example regarding the acquisition of data by a 3D scanner and the individual manufacture of prosthesis covers. Furthermore, it is not possible to modify or customize already existing prosthesis covers.

An object of the present invention is to provide a prosthesis cover and a process for producing it which enable customization or customized prosthesis covers.

This object is achieved by a prosthesis cover having the features of the main claim and a process having the features of the additional independent claim. Advantageous embodiments and developments of the invention are disclosed in the dependent claims, the description and the figures.

The prosthesis cover comprising at least one fastening device for fixing the prosthesis cover to a prosthesis component and a flexible main body having an inner side, which faces the prosthesis component in the placed-on state, and an outer side, which is situated opposite the inner side and on which the at least one fastening device is arranged, provides that at least one structural element is applied to the outer side in an additive manufacturing process and protrudes from the outer side. Using additive manufacturing processes, for example the different variants of 3D printing, it is possible to apply a multiplicity of different structural elements in different shapes, arrangements, designs and in different materials to a flexible, in particular elastic main body, in particular a textile, and fix them to a prosthesis component via the fastening device. The prosthesis cover or prosthesis cosmetic can consist of the main body and the fastening device. While conventional prosthesis covers make use of injection molded parts that can be arranged around the as-standard prosthesis components, this requiring a high investment expenditure for the tools, the proposed prosthesis cover makes it possible to customize, or individually produce prosthesis covers for a wide variety of prostheses and in a wide variety of shapes with low tool costs.

In one embodiment, the prosthesis cover is provided with multiple structural elements, which are applied at a spacing from one another on the main body. The structural elements may be arranged at different spacings from one another, in groups, in groups with different spacings from one another, and/or in groups with different spacings on the main body. The structural elements may have identical or different heights, which is to say protrude to different heights from the outer side. The individual groups may have structural elements with identical or different heights. Multiple groups with an identically formed height profile may be present. Similarly, it is possible for all the structural elements to be applied to the outer side at the same spacing from one another and form a height profile, which consists of structural elements with at least partially different heights.

The structural elements may be formed in regular and/or irregular shapes, for example as hemispheres, cones, pyramids, truncated cones, truncated pyramids, in droplet form, as plates, cubes, studs or other, arbitrary geometric shapes.

Owing to the variable arrangement and/or shapes of the structural elements, it is possible both to obtain an individual appearance and to accomplish desired functions such as protection, ventilation, haptics, etc.

In one embodiment, the structural elements are multicolored and/or made of different materials. In particular, the materials that adhere to the outer side of the main body are colored and have an opaque or only semitransparent form, while the layers of the structural-element material that are applied further away from the main body are transparent or at least semitransparent. In one variant, the structural elements have a full-color form. The outer side may also have a full-color and optionally opaque form. Owing to the different materials and/or different colorings, it is possible to easily achieve customization effects.

In one embodiment, the structural elements are made of an elastic material or comprise at least one elastic material or elastic material component, in particular the outer layers are or the outer layer is made of an elastic material. The outer layer is in particular transparent or semitransparent.

In one embodiment of the prosthesis cover, the material of the structural elements does not penetrate the main body, and therefore only the material of the main body can be felt on the inner side of the main body. In this respect, it is in particular advantageous if the main body consists of a material or comprises a material which has a high surface energy. Materials with a high surface energy facilitate the adhesion of the material of the structural elements, for example a resin, that is used for the additive manufacture. If use is made of textile main bodies or a main body having a textile content, materials with a high surface energy, for example polyamides or cotton wool, are preferably used. In particular in the case of textile components, the roughness of the raw materials or the hairiness of the yarn is of importance for the bond and the adhesion of the material of the structural elements. The larger the actual contact surface area between the material of the main body and the material of the structural elements is, the greater the adhesion of the material of the structural elements is.

In one embodiment, the main body of the prosthesis cover is elastic and/or retentive or absorbent for the material of the structural elements that is in contact with the main body, with the result that the deposited or printed-on material runs before the actual hardening operation and sinks into or permeates the main body. With preference, a polymer which is hardened by UV radiation is applied in the course of a 3D printing process. In one embodiment, the material is deposited onto a work platform via multiple nozzles and the still-liquid material is hardened, before it can penetrate the main body. For or after the hardening, the platform together with the main body is moved downward by the thickness of the deposited layer, or the printhead together with the printing nozzles is moved upward. Then, a further layer of the material to be deposited is deposited, in the desired shape and in the desired thickness, onto the already hardened layer and this is repeated until the desired shape of the structural element has been printed. The deposited material for the structural elements is advantageously liquid and is deposited by nozzles, with the result that it runs before the hardening operation. This makes it possible to obtain a very smooth surface of the structural elements, without reworking being necessary. In particular, the device for printing and depositing the materials has multiple printheads, and therefore multiple materials can be used and/or printing can be performed in multiple colors.

In one embodiment, the main body has multiple portions made of different or similar materials, in particular textiles, or a combination of textiles with for example other plastics materials, natural materials, cardboard, metals or composite materials, with the result that a mix of materials is produced or a main body can consist of one type of material in different embodiments. For example, in the case of a textile main body, use can be made of different materials of threads or yarns, different thread thicknesses, weave types, knit types, weave patterns, material qualities or colors and mixes of materials, which are then provided with the structural elements. Yarns or threads that can also be used are filaments and spun yarns; the filaments can be produced in different processes, for example in a melt-spinning process, a dry-spinning process and a wet-spinning process, optionally with subsequent texturing.

The various portions of the main body may consist of different materials or comprise material deviations, on which different arrangements and/or shapes of structural elements are placed.

In one embodiment, the main body has a proximal and/or distal holder or frame and can be arranged directly on the prosthesis component or a dimensionally stable part of the prosthesis, for example a tube or a joint component. The main body may also be arranged on a support, in particular a bowl-like support, for example a protector, with the result that the respective outer surface can be modified and customized via the fastening device. The fastening device may for example be an adhesive layer, which is adhesively bonded on the in particular bowl-like support. The support itself can also have a fastening device or a fastening element, in order to fix it to the prosthesis or a prosthesis component. The main body can be fixed detachably to the support and optionally to the holder or frame, for example via force-fitting elements such as magnets or form-fitting elements such as hook-and-loop closures, clamping devices, hooks, press studs, zip fasteners or the like.

In one embodiment, the holder is sewn, adhesively bonded, welded, integrally molded, or permanently fastened by overmolding on the frame, on the main body.

The process for producing a prosthesis cover comprising at least one fastening device for fixing the prosthesis cover to a prosthesis component and a flexible main body, in particular having a textile component, having an inner side, which faces the prosthesis component in the placed-on state, and an outer side, which is situated opposite the inner side and on which the at least one fastening device is arranged, provides that at least one structural element is applied on the outer side or to the outer side in an additive manufacturing process and protrudes from the outer side. The main body can be fastened by means of the at least one structural element to a holder or support, for example detachably via form-fitting elements or force-fitting elements or alternatively via an integrally bonded fastening means, for example by adhesive bonding or welding.

If the main body has a textile component, to increase the adhesion an embodiment of the textile component with plain weaves, such as an atlas weave, is advantageous since such plain weaves have a higher actual contact surface area than for example a twill weave in the case of a denim. If there are enough voids between individual stitches, knitted articles allow penetration of the material of the structural elements, in particular a hardenable resin, by the textile and very good adhesion to the main body. To increase the surface energy, the main body or the material of the main body may be coated, for example in the course of plasma coating or hydrophilization via surfactants.

In one embodiment, the main body comprises, instead of or in addition to a textile component, alternative materials such as cork, synthetic leather, leather, laminated materials, cardboard and also mesh materials, lattices or nonwoven material, in order to be able to provide further design effects by way of the main body and the main-body material. The prosthesis cover may also be applied on already-existing prosthesis components, for example adhesively bonded, fitted, held by a magnet, or placed on the prosthesis component or another prosthesis cover by another fastening device or other fastening means. The prosthesis cover may, in addition to a cover for prosthesis components for the lower limbs, also be intended for a prosthesis cover for prostheses for the upper limbs, for example as a cover for a forearm socket or as a prosthetic glove.

For a textile component, the material used comprises in particular a textured polyamide in the form of a knitted article, which in the best case scenario is single-knitted or -woven.

The structural elements are advantageously arranged at those locations of the prosthesis cover where they have an additional, in particular protective function, with the result that a material reinforcement at locations subject to particularly high loads can be achieved. The structural elements printed on in the additive manufacturing process additionally make it possible to have the effect of improved insulation, mechanical protection and/or improved cooling and also improved haptics and visual appearance of the prosthesis cover.

In one embodiment, the printed-on material of the structural elements is liquid or gel-like before the hardening operation and thereby makes it possible for the material of the main body to also be elastic. This makes it possible for the material of the main body to stretch before the printing operation and during the printing operation and to relax after the printing operation.

In one embodiment, the structural elements are spaced from one another far enough that the fundamental properties of the main body, in particular in terms of the flexibility and elasticity, are not or virtually not modified by the structural elements. A further advantage of the process and the prosthesis cover is that previous additive manufacturing processes have been used primarily to produce prototypes, and therefore the aspect of durability was not taken into consideration. With the existing process and the existing product, it is possible to achieve long-term durability of the structural elements of the product and be able to provide efficient and economic production of prosthesis covers. The connection of the applied material to the main body is durable; in particular up to now, in the printing of main bodies made of textile materials, the adhesion was unsatisfactory and the textile surfaces needed to be specially finished.

Exemplary embodiments of the invention are explained in more detail below on the basis of the figures. In the figures:

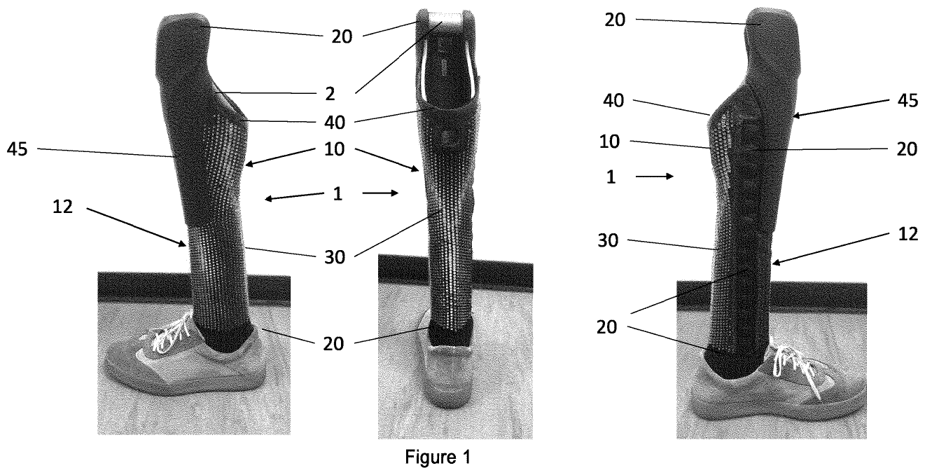

FIG. 1—shows three illustrations of prostheses comprising a prosthesis cover;

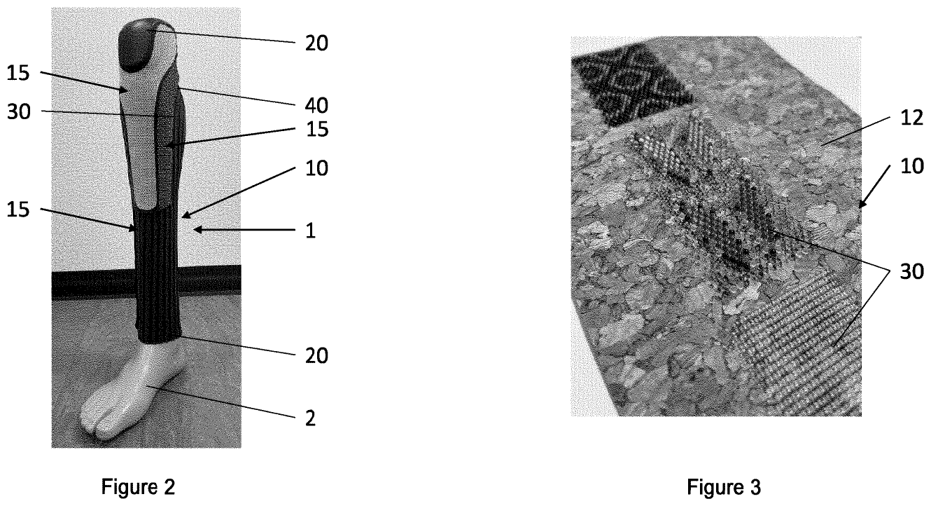

FIG. 2—shows a variant of the prosthesis cover;

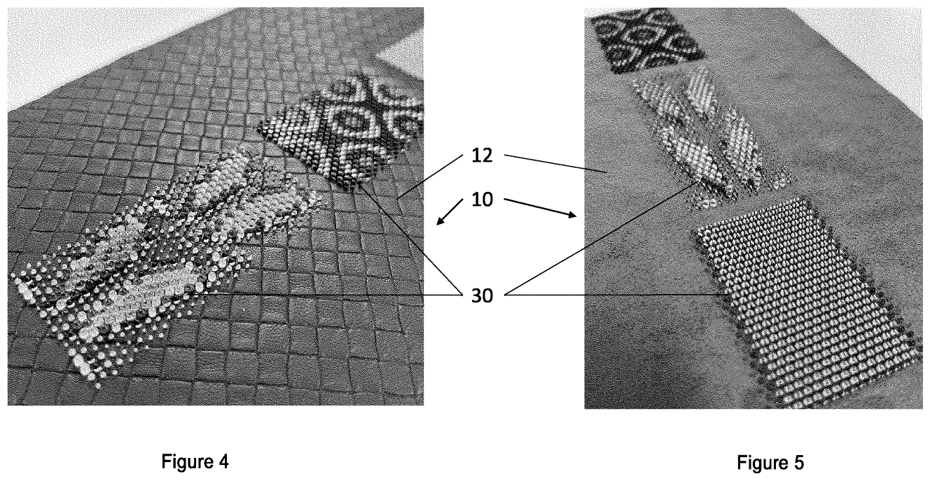

FIG. 4—shows an illustration of a detail of a main body with structural elements;

FIG. 4—shows a variant of FIG. 3 with a main body made of leather;

FIG. 5—shows a variant of FIG. 3 with a main body of building material; and

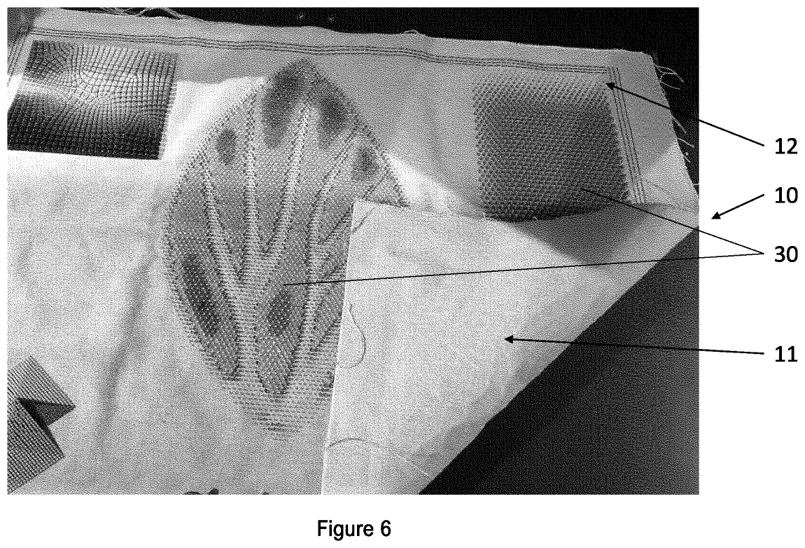

FIG. 6—shows a view of a detail of a main body with structural elements.

FIG. 1 illustrates three views of a prosthesis cover 1, from left to right in a lateral, posterior and medial view. The prosthesis cover 1 is fastened to a prosthesis component 2, which in the exemplary embodiment illustrated is in the form of a lower-limb prosthesis. The prosthesis cover 2 has a flexible main body 10 made of a textile material. The main body 10 may also be elastic. The prosthesis cover 10 is fastened to the prosthesis component 2 in the form of a prosthetic knee joint via a fastening device 20. The prosthetic knee joint has two lateral fastening parts or fastening points, with which the fastening device 20 interacts or engages. This fastening device is in the form of a cap arranged at the proximal end of the prosthesis cover 1. The cap has a bowl-like form and extends medially and laterally and also frontally over part of the prosthesis component 2. The fastening device 20 can be curved, or opened in another way, and placed around the prosthesis component 2. Distally adjoining the fastening device 20 is a support 45, which has a bowl-like form and extends distally in the region of the tibia. The support 45 extends approximately as far as the middle of the lower limb. From the support 45, a holder 40 or frame extends on the proximal, rear or posterior termination of the main body 10. The holder 40 is a flexible but dimensionally stable component which forms the proximal, posterior termination of the main body 10 and of the prosthesis cover 1. The main body 10 can be welded, sewn, adhesively bonded or clamped on the holder 40 or be durably or detachably fastened thereto in a different way. The main body 10 is also fastened to the support 40. In the exemplary embodiment illustrated, the main body is durably and non-detachably fastened to the support 45 on the lateral side, for example adhesively bonded, welded or integrally molded on the support or fixed thereto by form-fitting elements. As an alternative, the main body 10 is detachably fastened to the support 45, for example via form-fitting elements in the form of clips, hook-and-loop closures, hooks, undercuts or the like.

The right-hand illustration of FIG. 1 shows that fastening devices 20 are arranged extending from proximal to distal on the medial side of the main body 10. In the exemplary embodiment illustrated, the fastening devices 20 are in the form of magnets which are arranged on a lateral edge of the main body 10. The fastening devices 20 reversibly detachably fasten the main body 10 on the medial side to the support 45 and/or to the prosthesis device 2. The fastening is carried out in force-fitting fashion. As an alternative to the fastening devices 20 being in the form of magnets, the fastening devices 20 can also be in the form of zip fasteners, hook-and-loop closures or other form-fitting and/or force-fitting fastening devices. Distally in relation to the support 45, the initially open cross section of the main body 10 is placed on itself and closed, with the result that a substantially cylindrical and hollow basic shape, which tapers from proximal to distal and within which the prosthesis component 2 is arranged, of the prosthesis cover 20 is formed.

Also arranged or formed at the distal end of the main body 10 are fastening devices 20, in order to be able to fix the main body 10, for example to a prosthetic foot, a prosthetic foot cover or another component of the prosthesis.

As an alternative or in addition to the arrangement next to and behind the support 45, the main body 10 may also be arranged, in particular adhesively bonded on the outer side of the support 45.

In the illustrated exemplary embodiment, the main body 10 is made of a flexible and elastic textile, on the outer side 12 of which structural elements 30 are applied in the course of an additive manufacturing process. The outer side 12 lies on that side of the main body 10 that faces away from the prosthesis component 2. Structural elements in the illustrated exemplary embodiment in FIG. 1 are made of a transparent or semitransparent material and have different sizes. Both the diameters and the heights of the individual structural elements 30 are different, with some structural elements 30 having the same diameter and the same height. The structural elements 30 are provided in certain regions with different diameters and heights, in order to be able to provide a customized overall impression and improved functionality in regions subject to particularly high loads.

FIG. 2 illustrates a perspective overall view of a prosthesis cover 1 with a textile main body 10. Here, too, it is a prosthesis cover 10 for a lower-limb prosthesis with a prosthetic knee joint and a prosthetic foot. The prosthesis cover 1 is fixed at the proximal end, by means of the fastening device 20 in the form of a kneecap, to the prosthesis component 2 in the proximal region. A holder 40 also extends from the fastening device 20 in the posterior direction. The main body 10, in the fully placed-on state, circumferentially surrounds the prosthesis component in the lower-limb region. Depending on the configuration of the material of the main body 10, the prosthesis cover 1 can be drawn over the prosthesis component or is formed with an open cross section and can be closed via fastening devices, as has been illustrated in FIG. 1. In this exemplary embodiment, the main body 10 has multiple portions 15 made of different materials, which have different shapes, colors and functionalities. A first portion 15 may be formed with a different color than a second portion. A third portion 15 in the distal region has a particular flexibility and can be equipped with stabilizing devices or structural elements 30 in the longitudinal extent. Each portion has different structural elements 30 on the respective outer side, it being possible to arrange the support 45 behind the main body 10 in the tibia region for stabilization and shaping purposes. The portions 15 may be made of different materials. Different materials in this context are also similar materials, for example textiles, which have different properties. Different properties are created by different yarns, thread thicknesses, weave types or knit types and/or modified surface properties. There is also the option of providing a mix of non-similar materials, for example textile portions in conjunction with portions made of leather, plastic and/or metal.

FIG. 3 illustrates a view of a detail of a main body 10 made of a cork material, on the outer side 12 of which various structural elements 30 have been applied via an additive manufacturing process. The structural elements 30 have a droplet shape and are fastened on the outer side 12 at a spacing from one another. In the exemplary embodiment illustrated, three areas are provided with structural elements 30. A first area has purely transparent structural elements 30 which are at the same spacing from one another, have identical shapes and are arranged on the outer side 12. In the middle area are arranged droplet-shaped structural elements 30 in different diameters, with different heights and with different colors. At least one outer side or outer layer of the structural elements 30 is transparent. In the third region, structural elements 30 of the same type but different colors are applied to the outer side 12.

In FIGS. 4 and 5, the regions with the structural elements 30 are applied to different materials of the main body 10. The design of the structural elements 30 has remained the same; in FIG. 4, the material of the main body 10 is leather and in FIG. 5 it is a velour fabric.

FIG. 6 illustrates a main body 10 with an outer side 12 having structural elements 30, which have been applied to the outer side 12 in an additive manufacturing process. Part of the main body 10 has been turned up so that the inner side 11 of the main-body material can be seen. On the inner side 11, it can be seen that no material of the structural elements 30 has penetrated the main body 10, and therefore the inner side 11 of the main body 10 is free of the material of the structural elements 30.

FIG. 6 shows four different regions with different shapes, sizes, colors and different spacings of structural elements 30 from one another. Structural elements 30 of the same shape can have been applied in different colors to the outer side 12. At least one color layer, generally multiple layers of the material of the structural elements 30 that is applied layer by layer, are colored and are surrounded or covered by transparent or semitransparent further layers and an outer layer. Structural elements can have different spacings from one another and/or protrude from the surface of the outer side 12 to different heights.

The advantage of the configuration of the main body 10 with a flexible, in particular sheet-like main body is that it can be produced in customized fashion very easily in a corresponding manufacturing installation. For this, the main body 10 is tensioned on or fixed to a support frame and inserted into what is referred to as a 3D printer. The 3D printer operates on the polyjet 3D printing principle, which involves a hardenable polymer being deposited via a printhead with multiple nozzles onto the workpiece, in the exemplary embodiment a textile. The deposition is carried out in a liquid state through the respective nozzles. After being deposited from the nozzles, the still-liquid material of the structural elements is hardened, in particular via UV light, with both the viscosity of the deposited material and the duration of action and the moment of action of the UV light being coordinated such that although the first layer, which directly contacts the material of the main body 10, permeates the material, it does not penetrate it. In the case of a stationary printhead, then the support device for the main body travels downward by the thickness of a layer which has been or is to be deposited. A layer thickness is for example between 10 μm and 40 μm. After a sufficient period of hardening time, a further layer is deposited and is then hardened again. If use is made of multiple printheads or nozzles, printing can be performed in different materials and different colors. The printheads and nozzles can also be relatively movable in a plane parallel to the spanned areal extent of the main body 10, whether by way of a movable printhead or by way of a movable support. This makes it possible to obtain virtually any desired shapes of the structural elements. A computer program is used to control the application of advantageously photopolymer resin, which is present in corresponding cartridges in the printing device. It may be printed from multiple nozzles at the same time or else in succession. Each nozzle can be equipped with material having its own color different than the other colors, different hardness and other different properties, with the result that at the same time different colors and material properties can be applied and hardened. In one embodiment of the printed material, all colors can be created, for example via a CMYK system of color components, which are optionally mixed.

For each main body, it is possible to create different patterns, colors and materials individually by means of a computer program and apply them to the main body. Printing in the plane ensures a straightforward procedure. The main body may be printed already in the final form with which it is to be fitted to the prosthetic device. As an alternative, the required shape of the main body is cut out of a preferably rectangular base material. If appropriate, fastening devices, for example hook-and-loop closures or magnetic devices, are already present on the main body before the printing operation, with the result that the main body only needs to be placed around the prosthesis component. As an alternative, the main body can also be printed in the plane and then applied, for example adhesively bonded, clamped or reversibly fixed via hook-and-loop closures, to a support, for example an in particular bowl-like support or a protector. The fastening to the prosthesis component can also be carried out via the support, for example via clamping-in, latching-in, via a clip connection, via a screw connection or the like. The support is then also a fastening device or part of a fastening device.

The main body 10 with the structural elements applied thereto can be adhesively bonded to already-existing components of a prosthesis device or fixed thereto in another way. The adhesive layer is then the fastening device.

1. A prosthesis cover, comprising:
at least one fastening device for fixing the prosthesis cover to a prosthesis component;
a flexible main body comprising
an inner side, which faces the prosthesis component in a the placed-on state, and
an outer side, which is situated opposite the inner side, wherein the at least one fastening device is arranged on the outer side of the flexible main body, and
at least one structural element is applied to the outer side in an additive manufacturing process, wherein the at least one structural element protrudes from the outer side.

2. The prosthesis cover as claimed in claim 1, wherein the at least one structural element comprises multiple structural elements are applied at a spacing from one another on the main body.

3. The prosthesis cover as claimed in claim 1 wherein the at least one structural element comprises multiple that the structural elements or groups of structural elements arranged at different spacings from one another and/or with different heights on the main body.

4. The prosthesis cover as claimed in claim 1 wherein the at least one structural element comprises multiple structural elements are formed in regular and/or irregular shapes.

5. The prosthesis cover as claimed in claim 1 wherein the at least one structural element comprises multiple structural elements, wherein at least some of the structural elements are different in color from each other, or at least some of the structural elements are multicolored, and/or at least some of the structural elements are made of different materials from each other.

6. The prosthesis cover as claimed in claim 1 wherein the at least one structural element comprises multiple structural elements and wherein at least some of the structural elements are made of an elastic material.

7. The prosthesis cover as claimed in claim 1 wherein the at least one structural element comprises multiple structural elements and wherein at least some of the structural elements have a transparent outer layer or a semitransparent outer layer or a full-color outer layer.

8. The prosthesis cover as claimed in claim 1 wherein the at least one structural element is comprised of a material which does not penetrate the main body.

9. The prosthesis cover as claimed in claim 1 wherein the main body is elastic and/or is retentive for the material used in the at least one structural element that is in contact with the main body.

10. The prosthesis cover as claimed in claim 1 wherein the main body has multiple portions, wherein at least some of the multiple portions are made of different materials.

11. The prosthesis cover as claimed in claim 10, wherein the at least some of the multiple portions made of different materials have different arrangements and/or shapes relative to the at least one structural element.

12. The prosthesis cover as claimed in claim 1
wherein the main body has a proximal holder or frame and/or a distal holder or frame, and/or
wherein the main body is arranged on a support.

13. The prosthesis cover as claimed in claim 12, wherein the main body is adhesively bonded on the support, or wherein the main body is fixed detachably to the support by the at least one fastening device.

14. The prosthesis cover as claimed in claim 1 further comprising at least one holder for the main body, and wherein the at least one holder is sewn, adhesively bonded, welded, integrally molded, or overmolded on the main body.

15. The prosthesis cover as claimed in claim 1 wherein the at least one fastening device is in a form of a form-fitting element and/or force-fitting element.

16. A process for producing a prosthesis cover comprising at least one fastening device for fixing the prosthesis cover to a prosthesis component and a flexible main body having an inner side which faces the prosthesis component in a placed-on state, and an outer side situated opposite the inner side wherein the at least one fastening device is arranged on the outer side of the main body, comprising applying at least one structural element to the outer side in an additive manufacturing process such that the at least one structural element protrudes from the outer side.

17. The process as claimed in claim 16, further comprising fastening at least one holder or support to the main body with the at least one structural element.

18. The prosthesis cover of claim 1 wherein the at least one structural element comprises multiple structural elements, wherein at least some of the multiple structural elements may have the same or a different shape from each other, and wherein the multiple structural elements have shapes selected from the group consisting of hemispheres, cones, pyramids, truncated cones, truncated pyramids, in droplet form, as plates, cubes, and studs.
